(19) United States
(12) Patent Application Publication
(10) Pub. No.: US 2024/0254518 A1
(43) Pub. Date: Aug. 1, 2024

(54) SYSTEM AND METHOD TO RECYCLE THE WATER AND AMMONIA AND OPTIONALLY OTHER CELL MEDIA NUTRIENTS FOR A POWER-TO-GAS PLANT IN BIOLOGICAL METHANATION UTILIZING BIOCATALYST (METHANOGEN)

(71) Applicant: ELECTROCHAEA GMBH, Planegg (DE)

(72) Inventors: Zachary PINDER, Planegg (DE); Jose RODRIGO, Planegg (DE); Doris HAFENBRADL, Planegg (DE); Birgit LEWANDOWSKI, Planegg (DE)

(21) Appl. No.: 18/562,156

(22) PCT Filed: May 18, 2022
(86) PCT No.: PCT/EP2022/063412

§ 371 Date: Nov. 17, 2023

(30) Foreign Application Priority Data
May 18, 2021 (DE) 10 2021 112 887.0

(51) Int. Cl. C12P 5/02; C12M 1/00; C12M 1/107

(52) U.S. Cl. CPC C12P 5/023; C12M 21/04; C12M 29/04; C12M 29/18; C12M 47/18

(57) ABSTRACT

The present invention refers to a method to convert H2 and CO2 into methane by methanogenic microorganisms in a bioreactor in a continuous production process for methane enriched gas compositions, while recycling of at least one ammonia compound and/or recycling of electrons, wherein water (H2O) serves as the carrier for electrons. Metabolic water is removed to keep concentrations constant and purified for feeding to electrolyzer to generate hydrogen for supply to methanation reaction to reduce need for freshwater and ammonia to be supplied.

The state of the art for power-to-gas plant installations is to utilize water in an electrolyzer to produce H₂ and O₂ gas. The H₂ is fed combined with CO₂ in a methanation reactor to generate renewable methane. In this methanation reaction also “metabolic water” is formed and, together with the biomass, nutrients, and dissolved gases, is discharge into the sewer system as wastewater.

E.g., in a bioreactor within this process, a culture of methanogenic microorganisms utilize hydrogen to catalyse the methanation reaction as follows:

CO₂+4H₂→CH₄+2H₂O.

The water produced by this methanation process is also called “metabolic water” or “free water”. A problem associated with the production of metabolic water is the dilution factor of the medium components within the culture medium that must be specifically addressed. If the bioreactor is, e.g., a microbial electrolysis cell (MEC) this dilution factor is modified by the liquid migration processes that take place between the anodic and the cathodic chamber of the MEC which may be separated classically by a proton exchange membrane. The nutrient requirements of the methanogenic organisms are typically supplied as culture medium or as concentrated medium stock solutions in the prior art, which have to be added continuously in continuous or fed batch modes to guarantee a normal methanation rate during operation. This continuous addition of fresh medium stock is a significant detrimental part of the operational costs of the process.

At high volumetric productivities of the system (e.g., volume of methane/reactor volume/day, WD of >200) large amounts of metabolic water are generated. In the state of the art this metabolically produced water has to be continuously removed and discharged as wastewater or the wastewater is provided to off-site systems, e.g., into a sewer system for recovery and purification of nutrients.

Moreover, the utilization of water for large industrial power-to-gas plants has a cost and resource implication especially in countries where the access to fresh and clean water is limited and therefore cost-intensive. Thus, a recycling of (process) water within the system of large industrial power-to-gas plants especially water of high purity to significantly reduce the consumption of (pure) freshwater and additionally recycling of nutrients for the methanogens out of the classically discharged wastewater are of central interest. Of the possible nutrients to be recycled ammonia compounds are of special interest as they are firstly crucial for maintaining vital methanogens competent to produce methane and secondly cost-intensive to acquire when applied as new additives for the cell culture medium of methanogens.

Besides its role as a main component of the liquid culture medium needed for division, growth and maintaining of the methanogens and the medium where bio-methanation reaction occurs in the bioreactor, water has another highly important role, namely it serves as the source of electrons needed to generate H₂ to run the biomethanation process. Requirements for wastewater treatment are demanding in order to enable the recirculation of water to an electrolyzer to generate H₂ as intermediate electron carrier to be fed back to the bioreactor system. In state of the art this is generally performed via extensive downstream water cleaning means and multi-stage purification steps located off-site of the power-to-gas plants to gain water purified enough to fulfill the prerequisite to be used for the upstream electrolyzer. If the water to be recycled is not purified enough electrolyzers it increases the risk to be damaged by contaminants in the wastewater over time and thus their lifetime will be reduced at high acquisition costs.

It is thus an object of the present invention to overcome the described disadvantages of the state of the art, especially to optimize the bio-electrochemical processes under continuous operation conditions underlying the bio-electromethanation process and thus to provide a scalable, reliable and continuous production process for methane enriched gas compositions.

The object of the present application has been solved by the newly developed method as specified in claim 1 of the present invention.

In particular, to achieve the stated aim a method is provided to produce methane (biomethanation) in a bioreactor by utilizing a culture of methanogenic microorganisms in a culture medium for producing and collecting methane or a methane enriched gas composition the method comprising the steps of:
i. recycling of at least one ammonia compound and/or;
ii. recycling of electrons;
wherein water (H₂O) serves as the carrier for electrons;
including the steps of:
   a. extracting from the culture medium a metabolic water fraction comprising an at least one ammonia compound and the electron carrier water;
   b. separating the at least one ammonia compound and/or the water of the metabolic water fraction;
   c. isolating the separated at least one ammonia compound and/or the water, wherein the water is in the form of pure water.

The inventors of the present invention have advantageously and surprisingly found by running a bioreactor under such treating regime an at least one ammonia compound flow and/or a recycling of electrons flow is now used sensibly and fed back into the culture medium in the bioreactor via several steps within or on-site off the overall—e.g., power-to-gas plants-energy system. Via the inventive method pure water is generated allowing for recycling of otherwise wasted metabolic water into the electrolyzer, where the H₂ is produced and supplied again back to the methanogenic microorganisms in the cell culture medium by means of renewable energy and made available e.g., in in the circle for ongoing biomethanization. Thus, significantly reducing the consumption of freshwater to be supplied and consumption of the at least one ammonia compound to be supplied within the system.

“Metabolic water” according to the present invention refers to water or H₂O molecules, which are produced by the methanogenic organisms during metabolic activity and the process of methanogenesis.

Furthermore, it is an advantageous step of the method according to the present invention to remove, regularly or continually, excess moisture and/or an excess of metabolic water from the culture media thereby ensuring the correct concentration and/or dispersion of the nutrients in the media.

According to a further embodiment of the method of the present invention in step a) the removing of the metabolic water in the culture medium from the bioreactor is done continuously or discontinuously at certain time points instead of doing it continuously.

Ensuring a continuous methane production is a relevant feature of the present invention and an advantageous effect of implementing the steps of the method as described. According to the invention, methane is produced by methanogenic archaea from single strains or in mixed cultures, wherein a mixed culture is either a culture where a plurality of, therefore two or more, strains may also be employed, or a culture where a plurality of additional species interact with methanogenic archaea, or any combination thereof.

In the context of the present application, “methanation”, or “methanogenesis” or “bio-(electro)methanation”, is understood as the production of methane or a methane enriched gas composition as carried out by methanogenic microorganisms, such as those included in a list of methanogenic microorganisms suitable to carry out the present invention as described below.

In the understanding of the present invention, a “bioreactor” stands for a reactor, and is either a bioreaction vessel, or a bioreaction enclosure, or a bioreaction tank, and/or at least a bioreaction chamber, and/or a cell, or a combination thereof, as also intended in the state of the art, able to withstand variations of e.g. temperature and/or pressure, among others, and/or able to maintain whichever imparted values of e.g. temperature, and/or pressure are assigned or have to be maintained, before, after or during the reaction process, and wherein the intended reactions relevant for carrying out the invention may take place. Such reactions are understood as bioreactions as they pertain to the domain of reactions wherein microorganisms are involved, and herein referring to their normal physiology—such as, e.g., metabolic fermentation, or aerobic or anaerobic digestion—and that, as such, require suitable environments, suitable cultures of microorganisms, suitable culture mediums and suitable reactants to occur. A bioreactor in the meaning of the invention, performs reliably within the tolerance values of each variable in order to enable the method as disclosed, and it is expected to allow the listed steps to be carried out reliably over time.

A suitable reactor for culturing methanogenic microorganisms, may be, by means of example only, a shake tank bioreactor, a continuous stirred tank bioreactor, an intermittent stirred tank bioreactor, a hollow fiber membrane bioreactor, a bubble column bioreactor, an internal-loop airlift bioreactor, an external-loop airlift bioreactor, a fluidized bed bioreactors, a packed bed bioreactor, a photo-bioreactor, a trickle bed reactor, a microbial electrolysis cell, etc., and/or combinations thereof.

Moreover, in the understanding of the present invention, a suitable bioreactor may also include a “microbial electrolysis cell (MEC)”. Various suitable MECs are known in the state of the art and known by the practitioner. In more detail the microorganisms are cultured in a chamber at the cathode comprised in the MEC. The MEC may comprise a single compartment, or the cathodic compartment, or chamber, may be separated from the anodic compartment, or chamber, e.g., via a semipermeable membrane. The MEC has to be able to withstand variations of, e.g., temperature and/or pressure, among others, and/or able to maintain whichever imparted values of e.g., temperature, and/or pressure are assigned or have to be maintained, before, after or during the reaction process, and wherein the intended reactions relevant for carrying out the invention may take place. Such reactions are understood as bioreactions as they pertain to the domain of reactions wherein microorganisms are involved, and herein referring to their normal physiology—such as, e.g., metabolic fermentation, or aerobic or anaerobic digestion—and that, as such, require suitable environments, suitable cultures of microorganisms, suitable culture mediums and suitable reactants to occur. A MEC in the meaning of the invention, performs reliably within the tolerance values of each variable in order to enable the method as disclosed, and it is expected to allow the listed steps to be carried out reliably over time.

A MEC may comprise one or more sensors or components that measure and/or regulate values of, for example, (a) temperature, (b) pressure and/or (c) electrical potential difference, within a pre-set range. The values may be measured and/or regulated before, after or during the reaction process (e.g., methane production).

The operation mode of a bioreactor is classified as batch processes, fed-batch processes and continuous processes. According to the different embodiments of the method herein presented, a reactor may be chosen that most closely addresses the specific dynamics of a culture or the convenience by which methane is hereby extracted. In an embodiment of the present invention, a bubble column reactor, or a variant of it, such as an airlift bioreactor, or a continuously stirred tank reactor, and/or any of the above, may be used to conveniently carry out the method as described and a continuous culture is preferred, wherein near-balanced growth, with little fluctuation of nutrients, metabolites, cell number and biomass are observed.

In the understanding of the present invention “recycling” means that the perspective physically or chemical species/compound comprised in the extracted wastewater fraction that is to be recycled, e.g., the at least one ammonia compound and the electrons, respectively the electron carrier who is carrying the electrons is isolated and returned to the cell culture medium in the bioreactor. The at least one ammonia compound can be isolated and then optionally intermediately stored in a storage vessel in an “isolated form”, i.e., essentially pure with respect to chemical species contaminants others than water, i.e., in a purity of 10-98% or, i.e., in a purity of at least 70%, at least 75%, at least 80%, at least 85%, at least 90%, at least 95% or at least 98%. E.g., the at least one ammonia compound can be isolated and stored as pure anhydrous ammonia (NH₃) without water dilution, however, alternatively can be also diluted with water to various dilution percentages. The at least one isolated ammonia compound can be recycled back in the cell culture medium in an “isolated form”, i.e., in a purity of at least 70%, at least 75%, at least 80%, at least 85%, at least 90%, at least 95% or at least 98% or is diluted with e.g., (pure) water to the required amount (diluted form), e.g., to 25% and following then is recycled back in the cell culture medium.

The recycled water according to the present method has a purity of at least pure water to ultrapure water and such advantageously of high quality pure enough to feed back to an electrolyzer to regenerate H₂ to be fed to the methanogenic microorganisms. There are a variety of different analysis methods known in the state of the art to measure water purity well known to the artisan. It can be tested by looking at ion concentration by measuring electrical conductivity or resistivity, the later which is the reciprocal of conductivity. On-line monitoring of total organic carbon (TOC) may also be performed to validate the quality of water purification. Conductivity is expressed in microsiemens/cm (pS/cm) and is used to classify water with a large number of ions present. “pure water” according to the present invention is defined to have a conductivity at 25° C. of 600 to 0 or 500 to 0, preferred 300 to 0 or 200 to 0 or most preferred 100 to 0 pS/cm. “Ultrapure water” according to the present invention is defined to have a conductivity at 25° C. of 100 to 0 pS/cm, preferred 50 to 0 or 25 to 0 pS/cm, most preferred smaller than 0.1 pS/cm. The pure water received according to the method of the present invention can additionally be further purified in subsequent steps if needed before fed to a respective electrolyzer.

A “metabolic water fraction” according to the present invention means an aqueous fraction of and extracted from the cell culture medium comprising metabolic water.

The “metabolic water fraction” is supposed to be extracted in a continuous manner from the cell culture medium in order to ensure the correct reactor level, concentration, and/or dispersion of the nutrients in the medium. Alternatively, it can be done in a discrete manner. “Metabolic water” according to the present invention refers to water or H₂O molecules, which are produced by the methanogenic organisms during metabolic activity and the process of methanogenesis. Optionally the bioreactor comprises at least one sensor for liquid level measurement of the cell culture medium in the bioreactor, which is coupled with means to extract the metabolic water fraction from the cell culture medium. This arrangement allows for controlling and regulating of the amount of the metabolic water fraction to be extracted. This in turn advantageously allows for more operational flexibility and can be used to adapt the metabolic water fraction extracting process of excess metabolic water with the actual production of metabolic water in the culture medium driven by the methanogenic activity of the methanogenic microorganisms.

However, in an alternative embodiment it can be also generally extracted in a “discrete manner”, e.g., timewise extracted at different time periods, which occur, e.g., once, twice, thrice, and more or at least once, at least twice, at least thrice and more within 24 hours (/a day). Each extraction time period is followed by a time period, where no metabolic water fraction is extracted. The same or different durations for the extraction time periods may be applied, advantageously allowing for more operational flexibility and adapting the metabolic water fraction extracting with the production of metabolic water in the culture medium and thus with the metabolic activity of the methanogenic microorganisms.

“Methanogenic microorganisms” according to the present invention, or autotrophic methanogenic microorganisms may be anaerobic archaea or even recently classified aerotolerant archaea, either in pure strains, or in consortia with a plurality of, i.e., two or more, strains, or in mixed cultures wherein methanation may be also encouraged by syntrophic exchange across different species.

As used herein, the term “methanogenic” refers to microorganisms that produce methane as a metabolic byproduct.

The term “culture” as used herein refers to a population of living microorganisms in or on culture medium. When part of the MEC, the culture medium also serves as the electrolytic medium facilitating electrical conduction within the MEC.

According to an additional embodiment of the method of the present invention, the method further includes the step of:
culturing in the bioreactor the methanogenic organisms in a suitable liquid culture medium comprising minerals in a continuous process;
redosing at least one fraction of the isolated at least one ammonia compound into the bioreactor and/or recycling the pure water to an electrolyzer or other processes.

According to the present invention, the method herein disclosed is concerned with the culturing of methanogenic microorganisms in a “continuous process”, wherein such continuity is understood as continuity in the production of methane or at least another synthesis product by the methanogenic microorganisms (continuous operating process) and continuity in the culture, wherein no step of separating inactive terminal biomass from active members of the colony is required. It is instead encouraged that dead biomaterial is kept in the reactor together with the active members across several stages of growth, as it is found advantageous that said biomass or biomaterial provide further substrate for the active culture, intensifying nutrition availability. Thus, in some embodiments, the methanogenic microorganisms may be but not necessarily cultured with dead biomaterial inside the bioreactor for a certain period of time, at least 24 hours, 2 days, 3 days, 4 days, 1 week, 2 weeks, 3 weeks, 1 month, or more. In other words, the present invention can be performed under so called “cell retention conditions” as described in the international application PCT/EP2020/060979 to avoid—as this widely happens in classical culturing methods of the prior art—that substantial numbers of cells are continuously washed out of the reaction vessel. These washed out cells have to be replaced by further cycles of cell division and cell growth therefore by utilization of CO₂ and H₂ for the generation and growth of cells rather than for the generation of the aimed methane output. This is unfavourable for the efficiency of the system. Alternatively, and/or additionally there may be the option to supply a sufficient amount of new methanogenic microorganisms to compensate the amount of cells washed out if the MEC is running under no cell-retention conditions (see PCT/EP2020/060979). According to an embodiment of the method of the present invention the recycling of electrons further comprises:
performing a reductive power regeneration with the isolated pure water by electrolysing the pure water and recycling the regenerated electrons back in the bioreactor, wherein H₂ serves as an intermediate electron carrier.

The inventors of the present invention have surprisingly and advantageously found that via the present method it is possible to regenerate reductive power in form of high-quality pure or even ultrapure water which is pure enough to be fed to the electrolyzer which in turn produces H₂ as intermediate electron carrier. H₂ in turn can be recycled back to the culture medium in the bioreactor and fed to the culture of methanogenic microorganisms. Electrolysing may be done according to common state of the art methods regarding water electrolysis technologies, e.g., via alkaline water electrolysis; solide oxide electrolysis; microbial (cell) electrolysis; polymer electrolyte membrane electrolysis or other electrolysis methods, e.g., alkaline ammonia electrolysis.

Furthermore, it is an advantageous step of the method according to the present invention to continuously remove excess moisture and/or an excess of metabolic or so-called free water from the culture media (by removing a metabolic water fraction thereof) thereby ensuring the correct dilution and/or dispersion of the nutrients in the cell culture medium. This in turn is important for cell growth and division as well as for the bio-methanogenic activity of the methanogenic microorganisms.

According to another embodiment of the present method the step of culturing the methanogenic organisms further comprise:
controlling and regulating the concentration of the at least one ammonia compound in the culture medium to maintain the at least one ammonia compound concentration in the culture medium to be at a given amount of 0.001 to 1.7M or of 0.005 to 1.5 M, or of 0.01 to 0.75 M, or of 0.02 to 0.5 M, preferably between 0.005 to 0.3 M or 0.01 to 0.2 M.

The present invention is besides others characterized by a step of controlling the supply of the recycled ammonia compound and/or the (resulted) concentration of the ammonia compound within the cell culture medium. In this context, controlling is understood in the general common meaning of keeping under constant monitoring the parameters related to the culture and essentially measuring said parameters or status indicators, using common methodologies and measuring instrumentation known in the art, since it might not be sufficient to keep under constant monitoring and therefore only control this parameter of the culture; therefore, a further embodiment of the present invention comprises in particular regulating the nitrogen source concentration within the cell culture medium continuously. In the understanding of the present application, regulating is intended as actively maintaining a “given value” or a given value span for a parameter, e.g., the at least one ammonia compound concentration of the culture, by using appropriate means to do so.

A “given value” according to the invention may be a defined value with given tolerances, tolerances within the measurements system or tolerances due to the variability within the culture or due to the culture diversity, wherein said value is suitable for enabling methanation; or a given value may be a range of suitable values, which achieve the same effect on methanation as a given value.

The present invention can be performed under so called “separated supply regime” of the at least one ammonia compound and optionally also a sulfur source and/or inorganic carbon source—as described in the international application PCT/EP2021/074025 which is herewith incorporated by reference for this regime—to avoid as this widely happens in classical culturing methods of the prior art that substantial numbers of these chemical species are continuously washed out of the cell culture medium within the bioreactor.

According to another embodiment of the present method the step of culturing the methanogenic organisms further comprise:
keeping the culture conditions anaerobic or facultatively anaerobic;
optionally stirring the culture; and/or
keeping the temperatures in a range from 5° C. and 95° C.

According to the present invention, the step of culturing the methanogenic microorganism in the method to produce methane in a bioreactor further comprises: keeping the culture conditions facultatively anaerobic and/or anaerobic; optionally stirring the culture, wherein the stirring of the culture can be carried out regularly, in intervals, continuously, or keeping the soluble culture at least in a certain slow and constant movement; and keeping the temperatures in a range between 5° C. and 95° C.; preferably 15-65° C., 30-80° C., 32-62° C., 50-70° C. or around 62° C.

While the temperatures may vary according to the presence of selected microorganism species within the culture, each of which better thrive within set ranges of temperatures, for most of the methanogenic microorganisms increased temperatures are not detrimental, and they may even assist in optimizing cellular metabolism and thus metabolic turnover or even methanation. In an industrial process a temperature must be controlled by energetic regulation; in this regard it is to be considered a valuable feature to reduce energy expenditure by enabling temperature control.

Consequently, it is of substantial importance to balance the optimized culture temperature and the corresponding hydrogen solubility against the costs for energy input. Interestingly, the method of the present invention was found to be most efficient in a temperature range between 32° C. and 85° C., 50 to 70° C. or around 62° C. at atmospheric pressure. If according to some embodiments one or more steps of the method according to the invention are carried out in a pressurized atmosphere, then the pressure is chosen to be preferably up to 16 bar, alternatively up to 20 bar, alternatively up to 50 bar, alternatively up to 68 bar, alternatively up to 110 bar or even up to 420 bar.

For other temperature or pressure ranges hydrogen solubility can be used as comparative feature. Accordingly, the present invention also refers to a culturing process at pressures equal or between the range of 1 to 10 bar. High pressure, e.g., 16 bar, 20 bar, 35 bar, 40 bar or 60 bar and correspondingly, higher temperatures, which would allow the same hydrogen solubility as at a temperature range as above or between 32° C. and 85° C., 50 to 70° C. or around 62° C. at atmospheric pressure are also encompassed.

Methanogenic microorganisms, in general, may live and grow also in a plurality of other and even extreme temperature ranges up to and well above 100° C., e.g., 140° C.; accordingly, the above temperature range is an indication of a preferred range, but it is not to be understood as limiting the scope of the invention.

According to another embodiment of the present method at least one methanogenic microorganism is hydrogenotrophic and is selected from the group of Archaea or archaebacteria comprising of Methanobacterium, Methanobrevibacter, Methanothermobacter, Methanococcus, Methanosarcina, Methanopyrus or mixtures thereof.

As used herein, the term “hydrogenotrophic” refers to a microorganism capable of converting hydrogen to another compound as part of its metabolism. Classical hydrogenotrophic methanogenic microorganisms are capable of utilizing hydrogen (H₂) and an inorganic carbon source as CO₂ in the production of methane. However, according to an embodiment of the invention classical hydrogenotrophic methanogenic microorganisms according to the definitions as given above may be modified, e.g., by way of genetic modification to produce additionally other synthesis products as methane from H₂ and a carbon source, e.g., geraniol as described in Lyu et at., 2016.

According to a further embodiment of the present method the step of extracting from the culture medium the metabolic water fraction comprises the step of filtrating the at least one ammonia compound and the water from the culture medium and/or comprises the step of evaporating excess water from the culture medium and/or comprises the step of distillation. One not limiting way how this can be performed is disclosed in Example 1.

The means how to extract from the culture medium a metabolic water fraction from the culture medium in a running bioreactor is well known by a skilled person. By this excess (metabolic) water is removed from the cell culture medium to avoid diluting of the cell culture mediums components by an excess of water.

According to one embodiment the step of filtrating excess water away from the culture medium is performed by reverse osmosis using at least one semipermeable membrane for the at least one ammonia compound and the water in contact with the culture medium. By directly using reverse osmosis the methanogenic microorganisms are retained in the cell culture medium in the bioreactor thus resulting in extracting fractions of cell-free culture medium, i.e., essentially only water and the at least one ammonia compound. Additionally, an antecedent step of microfiltration can be avoided when directly using reverse osmosis, thus making the method procedure simpler. The technique and how to perform reverse osmosis are well known to the skilled person.

Separating the excess (metabolic) water from the aqueous cell culture medium by filtration, e.g., reverse osmosis results in a concentrated and recovered cell culture medium. With reverse osmosis, the dissolved compounds can be removed from water by using high pressure and nano-sized membranes. Advantageously, the at least one membrane semi-permeable for water and the at least one ammonia compound in contact with the culture medium is located in the proximity of a device, e.g., a tube which is in contact with the culture medium and is under a negative pressure resulting in a net efflux of water from the bioreactor. Using ultrafiltration according to the present invention, all cells are kept inside the reactor (cell retention) and only the excess water is removed from the soluble components of the cell culture medium.

Reusing this recovered medium will positively lower the amount of fresh medium stock required to run the bioreactor. Besides the cost motivation, there might also be a motivation from a legislative perspective to reuse the minerals in the excess water phase, since it might contain compounds at concentrations exceeding the limits set by the local authorities for discharge into wastewater treatment plants.

According to a further embodiment of the present method the step of filtrating the at least one ammonia compound and the water from the culture medium comprises the step of:
extracting fractions of cell-free culture medium by filtration and coincidentally concentrating minerals from the extracted fraction of culture medium by at least one filtration step consisting of: nanofiltration, ultrafiltration and reverse osmosis; and
optionally, storing the concentrated minerals in a separate container until redosing in the bioreactor; or
extracting fractions of cell-free culture medium by filtration through at least one porous membrane in contact with the culture medium, preferably having a pore size of 0.4 to 0.1 m, particularly preferably of 0.3 m; and
subsequently concentrating the minerals from the extracted fraction of culture medium preferably by at least one further filtration step, e.g., nanofiltration, ultrafiltration, reverse osmosis and/or by at least one evaporation and/or distillation step; and
optionally, storing the concentrated minerals in a separate container until redosing in the bioreactor.

When performing filtration through at least one porous membrane in contact with the culture medium, preferably having a pore size of 0.4 to 0.1 m, i.e., microfiltration, this results in extracting fractions of cell-free culture medium as cells are too big to pass the pores of the membrane. By directly using at least one more filtration step consisting of: nanofiltration, ultrafiltration and reverse osmosis the methanogenic microorganisms are also retained in the cell culture medium, however, an antecedent step of microfiltration is avoided, thus making the method procedure simpler. A porous membrane according to the present invention can be made of ceramic.

As stated before, in the state of the art there is a tremendous overuse of nutrients because of the re-dosing of nutrients according to the production and loss of nutrients because of discharge of metabolic water comprising nutrients. A cell and/or nutrient recycling mode without constant re-dosing, normally results in limitations for growth and metabolism of the biocatalyst and consequently in a drop in the methanation rate and loss of efficiency of the process.

The inventors of the present invention have advantageously found that a balanced and selective strategy overcomes the reported limitations of the state of the art. Examples to be integrated in the inventive method include: nutrient pre-dosing; (selective) media refreshment cycles; selective addition and/or balanced mixture of individual components; lysis of biocatalyst cells resulting in re-availability of the nutrients and control of biomass. Some of these strategies are already described in the international application PCT/EP2021/074025 and are herewith incorporated by reference.

According to a further embodiment of the present method the step of separating the at least one ammonia compound and/or the water of the metabolic water fraction comprises:
running at least one evaporation and/or distillation step, preferably under vacuum, wherein the at least one ammonia compound is vaporized preferably in form of gaseous ammonia (NH₃) and separated from the remaining metabolic water components; optionally wherein the heat for running the at least one evaporation and/or distillation step derives from the methanation process within the bioreactor;
optionally isolating the vaporized and separated at least one ammonia compound by redissolving the vaporized and separated at least one ammonia compound in an aqueous source or by liquifying the vaporized and separated at least one ammonia compound e.g., by passing the vaporized and separated at least one ammonia compound to a gas washer, condenser or bubble column or
optionally, isolating the vaporized and separated at least one ammonia compound by recapturing the vaporized and separated at least one ammonia compound by applying cool and/or pressure to it and
optionally, storing the redissolved, liquified or recaptured vaporized and separated at least one ammonia compound until redosing in the bioreactor.

According to an additional embodiment of the present invention the heat for running the at least one evaporation and/or distillation step derives additionally or alternatively from running of the electrolyzer. One not limiting way how this can be performed is disclosed in industrial scale—see Example 1. The filtered process water flows through a separation unit, e.g., a semipermeable membrane for water and the at least one ammonia compound via reverse osmosis, allowing a broad range of nutrients to be separated from the (metabolic) water fraction and recycled to the reactor in form of e.g., a “retentate”. This recirculation step allows a reduction of the otherwise permanently required nutrient additions or a much more targeted dosing of the still required nutrients. The gained so called “permeate” filtered through the semipermeable membrane is to the best knowledge of the inventors of the present invention free of most nutrients, only the at least one ammonia compound in form of NH₄OH and other species, e.g., small non-polar compounds cannot be retained by reverse osmosis and are still present in the permeate. The permeate may therefore be sent to downstream purification processes, e.g., to a vacuum distillation (or similar technology, e.g., an evaporation and/or distillation step) for further purification of the process water on the one hand and/or the at least one ammonia compound on the other hand.

In addition to the advantages mentioned above, the heat generated in the methanation process can optionally be integrated into the metabolic water fraction treatment to regenerate ultrapure water and to receive separated and optionally isolated at least one ammonia compound thus further significantly increasing the energy efficiency of the inventive method.

According to a further embodiment of the present method the step of collecting a methane enriched gas composition, further comprises:
passing the methane enriched gas composition to a gas washer, condenser, or bubble column to isolate the at least one ammonia compound from the methane enriched gas composition by redissolving the vaporized and separated at least one ammonia compound in an aqueous source or by liquifying the vaporized and separated at least one ammonia compound.

According to a further embodiment of the present method the isolated at least one ammonia compound is in the form of NH₃, NH₄OH, (NH₄)HCO₃, (NH₄)₂SO₄ or NH₄Cl or combinations of the aforementioned. Preferably it is in the form of NH₄OH. To recover the at least one ammonia compound in form of NH₄OH has the advantage that it can be directly applied to the cell culture medium when needed and has some advantages versus storing it in form of a salt because the counter anion of such a salt will lead to an increase of the concentration of that counter anion in the cell culture medium. This in turn may influence parameters as salinity, nutrient availability, and cell growth, cell division and metabolic activity, particularly methanogenic activity of the exposed methanogenic microorganisms within the cell culture medium in the bioreactor. The counter ion also increases costs considerably when compared to the metabolically produced water. It also has the advantage of utilizing readily available pumping equipment that is already calibrated and placed in the proper injection point. Furthermore, the NH₄OH can be concentrated or diluted to the required amount.

According to an embodiment of the present method the at least one ammonia compound in the removed metabolic water fraction is NH₄OH, which is vaporized to NH₃ and then recovered as isolated NH₄OH.

According to an embodiment of the present method the method includes the step of:
recycling at least one fraction of the isolated at least one ammonia compound into an electrolyzer, wherein the at least one ammonia compound is NH₄OH and the electrolyzer is an alkaline ammonia electrolyzer.

According to an embodiment of the present method further comprising:
separating at least one entity of the minerals comprised in the metabolic water fraction from the remaining metabolic water components;
optionally storing the separated at least one entity of minerals; and
redosing of the at least one entity of minerals in the bioreactor.

The new recirculation process where pure water, the at least one ammonia compound and other nutrients are recovered and recycled positively enables the biomethanation cycle to be efficiently closed in one intern system.

According to an embodiment of the present method the at least one entity of minerals is selected from the group consisting of iron, nickel, potassium, phosphorus, sodium, chloride, cobalt, selenium, tungsten, magnesium, molybdenum, sulfur, tetrasodiumiminodisuccinate, nitrilotriacetate, nitrilotriacetic acid, L-cysteine and resazurin, chelators as, e.g., methylglycine-N,N-diacetic acid, citric acid, or mixtures of the aforementioned.

According to an embodiment of the present method the step of culturing the methanogenic organisms further comprise the steps of:
isolating the methanogenic microorganisms comprised in the extracted metabolic water fraction from the remaining metabolic water components;
optionally storing the separated methanogenic microorganisms;
optionally lysing at least fractions of the separated methanogenic microorganisms; and
recycling the separated methanogenic microorganisms and/or optionally lysed fractions thereof back in the culture medium.

As mentioned before, lysis of the biocatalyst cells, i.e., of methanogenic microorganisms results in re-availability of the nutrients and control of biomass.

According to an embodiment of the present method the step of culturing the methanogenic organisms comprises at least one cycle of culturing the methanogenic microorganisms under:
a first phase in a continuous process in a suitable liquid minerals containing culture medium comprising a reduced supply of at least one entity of minerals; followed by
a second phase, characterized by refreshing the culture medium;
optional followed by a third phase in a continuous process comprising a reduced supply of at least one entity of minerals.

A “at least one mineral” according to the present condition within the first phase and/or third phase refers to typical minerals, which are present in classical cell culture mediums, e.g., a nitrogen source (e.g., an at least one ammonia compound) and/or salts. According to one embodiment the “at least one mineral” is a nitrogen source. According to another embodiment the “at least one mineral” is a salt, e.g., a chloride containing salt. The chloride can be present in the salt respectively dissolved as saline solution as the anion of NaCl, MgCl, KCl, NH4Cl or any other suitable chloride salt known to the skilled person. The “at least one mineral” which supply is decreased may be the same or be a different one in the first and the third phase.

A “refreshing of the culture medium” according to the present invention within the second phase can be realized by changing the cell culture medium at least partly or by adding at least one nutrient, which triggers cell division and cell growth. Nutrients, which trigger cell growth and cell division are well known by an artisan and include the addition or the increase of a nitrogen source, e.g., an at least one ammonia compound, a sulfur source, phosphorous and cell growth factors. A combination of the described options for refreshing of the culture medium is also a possible option according to the present invention. This second phase can optionally be followed by a third phase, wherein the cells are again cultured in a continuous process comprising a reduced supply of at least one mineral. Then, the second phase is a transition phase flanked between two phases in a continuous process within the at least one cycle. Such a “refreshing of the culture medium” may be but not necessarily be applied every month, every half year for at least one day or at least one day to five days or at least one day to four days at least one day to three days.

According to an embodiment of the present invention additional nutrients are supplied to the cell culture medium continuously depending on the need of the cultured cells and the consumption of nutrients by the cells in a continuous process.

According to an embodiment of the present invention the step of culturing the methanogenic organisms comprises at least one cycle of culturing the methanogenic microorganisms under:
a fourth phase under cell retention conditions; followed by
a fifth phase, characterized by culturing the cells under no cell retention conditions;
optional followed by a sixth phase under cell retention conditions.

A “culturing the cells under no cell retention conditions” according to the present invention within the fifth phase refers to conditions in a running bioreactor, which does not enable and does not guarantee that cells, i.e., the methanogenic microorganisms are kept inside the bioreactor, i.e., methanogenic microorganisms will be washed out of the bioreactor during this phase. This fifth phase can optionally be followed by a sixth phase, wherein the cells are again cultured under cell retention conditions. Then, the fifth phase is a transition phase between two phases under cell retention conditions within the at least one cycle.

The inventors of the present invention have found, that culturing the methanogenic microorganisms under such no cell retention conditions may be advantageous at a certain running time of the reactor. This phase under such no cell retention conditions may promote cell division and cell growth, which may have a positive effect on the overall methanation process efficiency. Such no retention conditions may be but not necessarily be applied every month, every half year for at least one day or at least one day to five days or at least one day to four days at least one day to three days.

According to a further embodiment of the present invention wherein the method alternatively comprises:
collecting methane or a methane enriched gas composition and/or at least one other synthesis product from the bioreactor.

I.e., according to another embodiment of the invention a method is provided to produce and collect a synthesis product other than methane. According to a further embodiment a method is provided to produce methane and at least one other synthesis product and then to separately collect methane, or a methane enriched gas composition and the at least other synthesis product from the bioreactor.

According to a further embodiment the at least one other synthesis product different from methane or different from the methane enriched gas composition is selected from the group consisting of geraniol, vitamin A, cholesterol, carotenoids, and natural rubber.

### REFERENCES

Lyu Z, Jain R, Smith P, Fetchko T, Yan Y, Whitman WB (2016) Engineering the autotroph for geraniol production. ACS Synth Biol 5:577-581.

### SHORT DESCRIPTION OF THE FIGURES

FIG. 1: Analysis of nutrient concentration in a) cell suspension, b) cell-free cell culture medium, c) cell biomass (methanogenic microorganisms). Nutrient concentration in cell retention mode but without nutrient recovery. Vertical coordinate left (y-axis): Absolute concentration amounts based of standard media; Horizontal coordinate (x-axis): Analysed compounds

FIG. 2: Evaluation of distillation of fractions of cell-free culture medium (microfiltered medium). Cells are kept back in the reactor with a cell retention membrane (microfilter); filtered cell-free extracted metabolic water fraction. FIG. 2A Vertical coordinate left (y-axis): WD (volume of methane/reactor volume/day), [L/L/d]; Horizontal coordinate (x-axis): running time [h].

FIG. 2B Vertical coordinate left (y-axis): OD600 (optical density at a wavelength of 600 nm) as indicator for amount of biomass; Horizontal coordinate (x-axis): running time [h].

FIG. 3: A. Reactor set up (industrial scale derived from lab scale reactor experimentation) for removal of excess metabolic water under cell retention conditions by using a microfilter located in the cell culture medium in the bioreactor and using reverse osmosis filter outside of the bioreactor. Variant with vacuum distillation unit and condenser for clean pure water and gas wash for stream with NH3. Herein is: 1. Methanation Reactor, 2. Microfilter, 3. Discharge/Feed, 4. Separation, 5. Permeate 6. Vacuum distillation unit, 7. Retentate, 8. Condenser, 9. Clean water to electrolyzer, 10. Distillate stream with NH3, 11. Gas wash, 12. Product gas stream, 13. Captured NH4OH to concentrate, 14. Concentration Container, 15. Redosing, 16. To downstream processing, 17. Excess reactor heat utilization.

### Experimental Set-Up (Lab Scale)

A lab scale reactor is supplied with H₂, generated by an electrolyzer, and CO₂, a byproduct of biogas purification. The flow rates of hydrogen and carbon dioxide were adjusted to a 4.1:1 ratio. The temperature of the culture was 62.5° C. Metabolic water fraction was removed from the reactor and passes through a R/O membrane to remove the produced metabolic water and retain the metal/salts and biocatalyst. NH3 containing metabolic water flows to the vacuum distillation unit where excess metabolic heat is exchanged to keep the temperature favorable to remove the NH3 as gas. This is passed through a gas wash system, along with the product gas flow that also contains NH3 gas. The discharge of the gas wash is sent to the concentrating vessel to be concentrated to the appropriate concentration for redosing. The water in the vacuum distillation vessel also is fractionally removed and condensed to be sent to the electrolyzer.

B. Reactor set up for removal of excess metabolic water under cell retention conditions by using a biomass separation (microfilter) located in the cell culture medium in the bioreactor and using nutrient recovery unit (reverse osmosis filter) outside of the bioreactor. Variant with distillation unit and condenser.

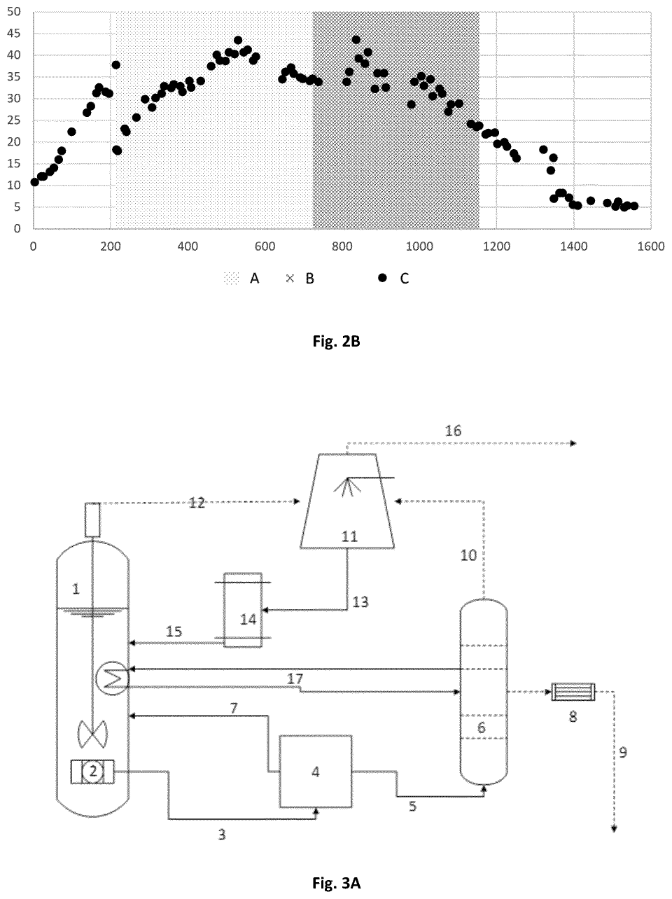

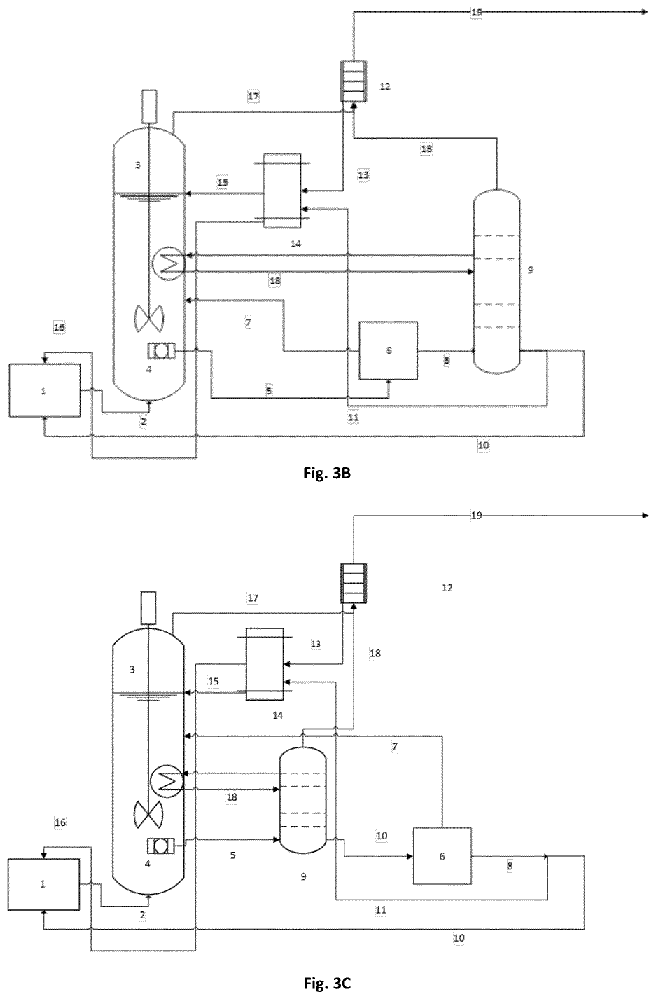

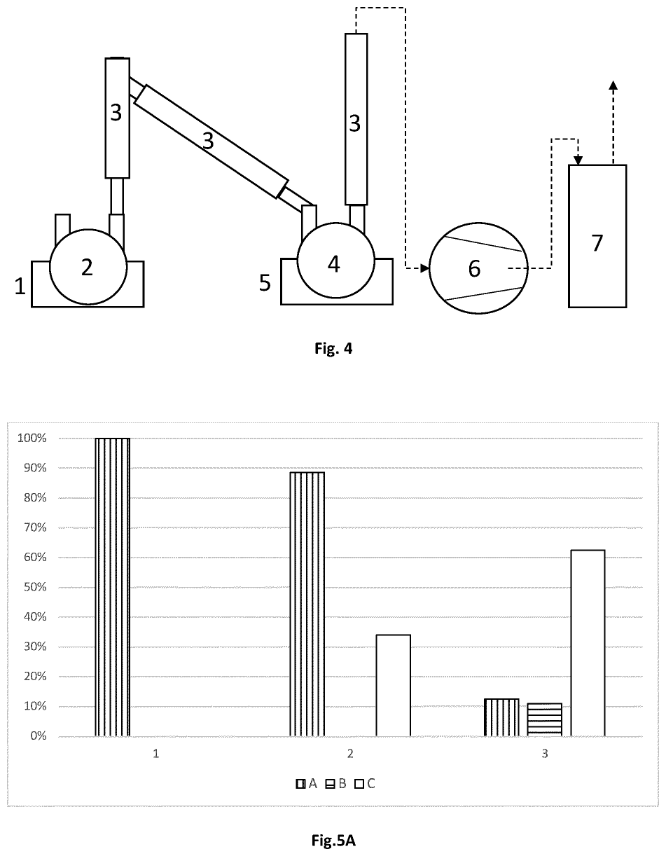

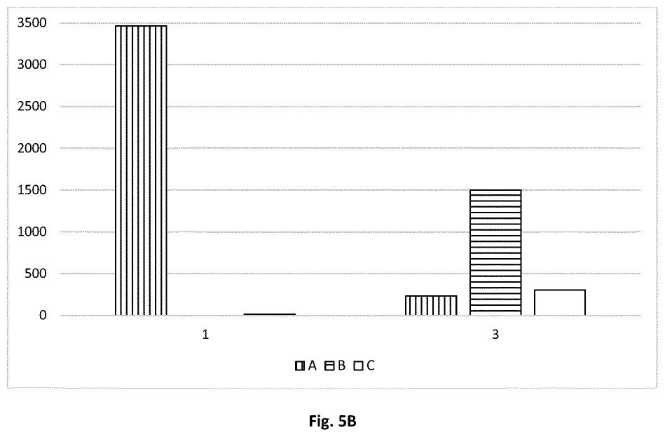

C. Same as FIG. 3B except evaporation (pressurized chamber) is placed before salt separation. Herein is: 1.Hydro/Alkaline Electrolyzer, 2. H₂ feed to reactor, 3. Methanation reactor, 4. Biomass separation, 5. Feed to recycling, 6. Salt recycling unit, 7. Retentate of salt, 8. Permeate, 9. NH3 evaporation chamber, 10. Water for electrolyzer, 11. Water for NH4OH recapture, 12. Condenser, 13. Captured NH4OH to concentrate, 14. Concentration container, 15. Redosing, 16. Evaporated NH3 stream, 17. Product gas stream, 18. Excess reactor heat utilization, 19. To downstream processing.

FIG. 4: Experimental set up for recycling of pure water according to the reactor set-up in FIG. 3A. Herein is: 1. Hot oil bath to keep 2 stable at 65° C.; 2. ,,Feed″ sample (microfiltered and subsequent reverse osmosis filtered metabolic water fraction), Round bottom flask; 3. Condensator; 4. “Condensate” sample, Round bottom flask; 5. Cool water bath to keep 4 at 10° C.; 6. Vacuum pump at 200 mBar(g); 7. “Gas” sample. Gas wash bottle with gas sparging fitting

FIG. 5: A. The amount of ammonia measured at each measurement point during the progression of the experiment as set up in FIG. 4. Values are shown as percent of total ammonia (mg) normalized to sample point's volume. Legend: 1) Start 2) Middle 3) End of experiment; A) Feed (microfiltered and subsequent reverse osmosis filtered metabolic water fraction) B) Concentrate C) Gas Wash; (1—left graph panel) One can see that at the beginning of the experiment 100% of the total ammonia is present in the feed vessel. By the time of the second sample (2—middle graph panel), it is seen that ammonia has left the feed vessel and been captured in the gas wash (3—right graph panel).

B. The graphic shows the conductivity in pS/cm of each analyzed aqueous sample measured at the beginning and end of the experiment as set up in FIG. 4 in each indicated sample vessel. The feed sample is reduced to 235 pS/cm, which is a normal range for tap water and can be directly feed to an electrolyzer's water purification unit. Legend: 1) Start, 3) End of experiment; A) Feed (microfiltered and subsequent reverse osmosis filtered metabolic water fraction) B) Concentrate C) Gas wash;

### EXAMPLES

The following examples illustrate viable ways of carrying out the described method as intended, without the intent of limiting the invention to said examples.

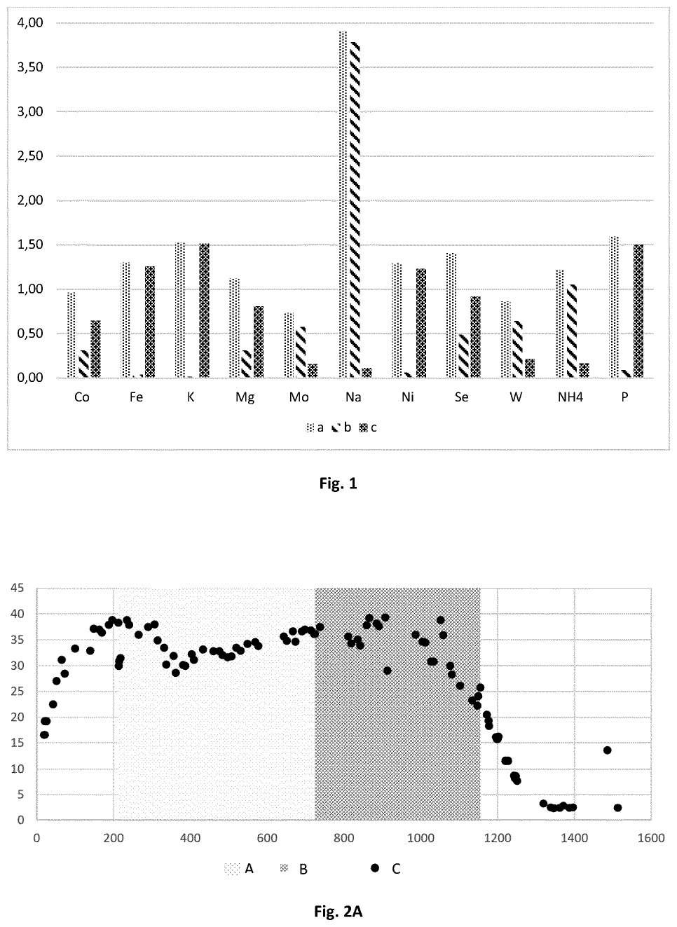

### Example 1: Analysis of Nutrient Concentration in a) Cell Suspension, b) Cell-Free Cell Culture Medium, c) Cell Biomass (Methanogenic Microorganisms) (FIG. 1)

The inventors of the present invention have set themselves the task to provide a method to convert H₂ and CO₂ into methane by methanogenic microorganisms in a scalable, reliable, and continuous production process for methane enriched gas compositions while recycling factors as nutrients important for culture and methanogenic activity of methanogenic microorganisms. Initially, therefore, the inventors have tested what the outcome with respect of nutrient concentration is in a) cell suspension, b) cell-free cell culture medium, c) cell biomass (methanogenic microorganisms) when culturing the methanogenic microorganisms by continuous addition of fresh nutrients/cell culture medium and under cell retention conditions and continuous addition of fresh nutrients/refreshing the cell culture medium (i.e., without nutrient recovery and nutrient recycling). The outcome is depicted in FIG. 1.

The experimental set-up was done as described in PCT/EP2020/060979 in Example 1 & 2.

### Example 2: Analysis of Distillation as a Potential Recovery System for Washed-Out Nutrients to be Recycled to a Culture of Methanogenic Microorganisms in a Running Bioreactor

In subsequent experiments the inventors of the present invention analyzed if a distillation method would be appropriate as a potential recovery system for washed-out nutrients to be recycled to a culture of methanogenic microorganisms in a running bioreactor.

For this, methanogenic microorganisms were kept in the reactor with a cell retention membrane (under cell retention conditions). The washed-out cell culture medium was collected and distilled and the received concentrated solution was recirculated to the culture of methanogenic microorganisms in the running bioreactor. This procedure was repeated once after the first recirculation of the distilled concentrated medium (2^{nd} recirculation). FIG. 2A, B shows the results. As can be seen the first recirculated distilled concentrated medium led to no significant change of metabolic performance as indicated via WD/time (FIG. 2A, area A) or cell mass (FIG. 2B, area A) in the running bioreactor over indicated time. However, after addition of the second recirculated distilled concentrated medium dramatically decreased cell mass (FIG. 2B, area B) and in parallel led to a significant drop in metabolic performance (FIG. 2A, area B). Indicating, that a continuous recovery system via repeated distillation of cell free washed-out cell culture medium is not sufficient to apply in a running bioreactor utilizing methanogenic microorganisms.

### Example 3: Analysis of the Experimental Set Up According to FIGS. 3A, B, and C, by Applying a Special Filtration System of Reverse Osmosis Subsequent a Microfiltered Washed Out Cell Free Cell Medium and Subsequent Recirculate the Concentrated Nutrients

The inventors observed that via such a nutrient recycling system did not lead to a significant change of metabolic performance as indicated via WD/time or cell mass change in a running bioreactor over a certain time period as compared in a normal cell retention condition mode with continuous addition of fresh nutrients/cell culture medium, thus indicating a highly useful way to reduce loss of nutrients.

### Example 4: Analysis of the Experimental Set Up According to FIGS. 3A, B, and C. Complete Recycling System

Additionally, the inventors were interested to provide a system to recover and recirculate the at least one ammonia compound which could not be retained via reverse osmosis with the other minerals. Moreover, the inventors wanted to allow for recovery and recirculation of water pure enough to be fed to a subsequent electrolyzer to recycle H₂ back to the cell culture medium comprising methanogenic microorganisms, utilizing H₂ for methanogenesis.

The new recirculation process enables the biomethanation cycle to be efficiently closed. The requirements for wastewater treatment are demanding in order to enable the recirculation to the electrolyzer, so a multi-stage process has been developed for this purpose. The following is a detailed description of the process based on two exemplary possible experimental set-ups as depicted in FIG. 3A, 3B, 3C in simplified form.

FIG. 3A:
1) At high volumetric productivities of the system (e.g., WD of >200) large amounts of metabolic water are generated. This metabolically produced water has to be continuously removed and discharged into the sewer system for purification. This flow is now used sensibly by the new process and fed back via several steps into the electrolyzer, where the H2 is produced again by means of renewable energy and made available for the methanization process.
2) A microfilter unit enables the retention of the microbial biomass, which would otherwise also be discharged via the process water and would no longer be available for the biocatalytic activity.
3) The filtered process water flows through a Separation unit (4), allowing a broad range of nutrients to be separated from the process water and returned to the reactor via the retentate (7). This recirculation step allows a reduction of the otherwise permanently required nutrient additions or a much more targeted dosing of the still required nutrients. These steps can eventually be combined by retaining the biocatalyst directly via the applied separation step.
4) Separation unit.
5) The permeate is free of most nutrients, only NH3/NH4, ammonia compounds and others cannot be retained by the separation unit (4) and are still present in the permeate. The permeate is therefore sent to a vacuum distillation (or similar technology) unit (6) for further purification.
6) The heat (17) for the operation of the (Vacuum-) distillation unit comes from the heat generated in the methanation process (1). Through the distillation process, the volatile compounds still contained can be separated and pure water vapor is produced as a product, which can be liquefied by a condenser (8) and fed (9) to the electrolyzer. Highly volatile compounds such as NH3 leave the distillation unit via (10). NH3 is fed to a gas scrubber and dissolved in water again. Via (13), the NH3 is concentrated in a tank and fed back to the reactor as required. Here, too, savings potentials can be expected with regard to the continuously required NH3 addition. Similarly, the product gas (12) of the reactor (1) is to be passed through the gas scrubber (11) in order to recover NH3 lost via the product gas. The components that are not soluble in the gas scrubber will be fed to downstream processing via (15). H2 and H2S are the two main components to be removed via such downstream processing. Depending on the gas feed there are also a list of contaminates. The downstream processing can be a combination of knockout tanks, chillers, heaters, filters, scrubbers, and membranes.

The potential use of the purified water to be recycled back to the concentration vessel to achieve the proper dilution required (11) as well as recycling back to the electrolyzer itself (10) is shown in FIG. 3B. As in FIG. 3A it also depicts the use of a condenser (12) utilizing already present and required equipment of the plant as well as the return of NH3OH to the hydrogen generator, e.g., alkaline ammonia electrolyzer (16). FIG. 3C shows the exchange in positioning of separation unit (6) with evaporation (distillation) unit (9). This gives the benefit of utilizing the advantageous pressure and temperature conditions for more selection of NH3 gas. This is different from the variant in Example 2 (1) as there is no vacuum, thus causing side reactions.

### Example 5: High Recovery of the at Least One Ammonia Compound and High-Quality Water can be Recovered Pure Enough to be Fed to an Electrolyzer

Experimental set up as depicted in FIG. 4.

### Methods

Unless otherwise stated, all equipment was supplied by Carl Roth GmbH+Co. KG, Karlsruhe, Germany.

FIG. 4: A glass 500 mL 4-neck round-bottom flask (feed vessel), 2 was connected to a second glass 500 mL 4-neck round-bottom flask (concentrate vessel), 4 in series using one 160 mm spiral condenser vertically and one 400 mm Liebig condenser to the concentrate via a ,,Y″ adapter and 1050 delivery adapter. A second 160 mm spiral condenser was attached to the outlet of the concentrate vessel 4. The outlet of this condenser was attached to a vacuum pump using a vacuum adapter and vacuum resistant tube. The outlet of the vacuum pump was attached to a gas wash bottle (gas wash) 7, filled with 500 mL deionized (DI) water. Thermometers with adapters were placed in both feed 2 and concentrate 4 and a stir bar in the feed vessel 2. Any open necks of the flask were closed with stoppers. All glass necks were sealed with vacuum grease. The feed vessel 2 was placed in a hot oil bath so that internal temperature was 65° C. The concentrate vessel 4 was placed in a cooling bath so that internal temperature was 10° C.

300 mL reverse osmosis permeate from an actively operating bioreactor was placed in the feed vessel 2. Samples were taken from the gas wash vessel 7 and feed vessel 2, then vacuum pump turned on, regulated to 100mBar. After 1 hour, the vacuum was stopped, and a 1 mL ,,middle″ sample was taken from the feed 2 and gas vessel 7.25 mL DI water was added to the concentration vessel 4 then the vacuum was then restarted. After two hours the experiment was stopped and ,,end″ samples were taken from the 3 sampling points. Samples were stored with 30 μL 1M H2SO4 at 5° C. overnight then analyzed using a Amplite™ colorimetric ammonia quantitation kit (AAT Bioquest, Sunnyvale, California, USA).

Results concerning ammonia recovery are depicted in FIG. 5A. At the beginning of the experiment 100% of the total ammonia is present in the feed vessel 2. By the time of the second sample (middle), it is seen that ammonia has left the feed vessel 2 and been captured in the gas wash 7. There is a discrepancy between the percentage transfer, with too much being in the gas wash 7 but according to the best knowledge of the inventors this is thought to be based on measurement inaccuracies. Still, qualitatively the result shows the migration of the ammonia compound.

At the end point sampling, only 13% ammonia is left in the original feed vessel 2, with significantly high 62% recaptured in the gas wash 7 and 11% captured in the concentrate sample 4. This shows that at least 87% of the original ammonia is removed and 73% is captured to be utilized for recirculation, with only 14% missing likely pushed through the gas wash.

Additionally, the amount and the purity of the water was analyzed running the experimental set-up as depicted in FIG. 4. Results concerning the amount of recovered water are depicted in FIG. 5b. The graphic shows the conductivity in pS/cm measured at the beginning (Feed vessel: 3467; Concentrate vessel: 0; Gas wash vessel: 14) and end of the experiment (Feed vessel: 235; Concentrate vessel: 1500; Gas wash vessel: 306) in each sample vessel. The feed sample is then reduced to 235 pS/cm, which is a purity sufficient to be directly feed to an electrolyzer's water purification unit. The conductivity was measure once at each sampling point using a handheld meter (HM Digital)

### Example 6: Analysis of Life-Cycle Assessment (LCA)—Global Warming Potential Calculations Using the Inventive Method

A product carbon footprint is a mean to measure of direct and indirect greenhouse gas (GHG) emissions associated with all activities in the goods life cycle. A life-cycle assessment (LCA) can be used to calculate such carbon footprints. LCA focusses on, e.g., GHG emissions that have an effect on climate change or the global warming potential (GWP) itself. Based on the experiments the inventors of the present invention have performed, a 73% reduction of external ammonia dosing by using recycled at least one ammonia compound results in a 27% reduction of GWP, with a complete reduction in external ammonia dosing at least 35% reduction of GWP is possible. Thus, regarding the LCA, the inventors of the present invention calculated a maximum of 35% reduction. This assumes a positive 100% reduction in NH3 dosing. The reuse of cleaned water to the electrolyser results in a ca. 22% reduction of freshwater use. Thus, the above experiments already demonstrate how the inventive recycling system and method for recycling the at least one ammonia compound and electrons have a beneficial impact on the GWP of the technology.

1. A biomethanation method in a bioreactor utilizing a culture of methanogenic microorganisms in a culture medium for producing and collecting methane or a methane enriched gas composition comprising the steps of:
i. recycling of at least one ammonia compound and/or;
ii. recycling of electrons;
wherein water (H2O) serves as the carrier for electrons;
including the steps of:
a. extracting from the culture medium a metabolic water fraction comprising an at least one ammonia compound and the electron carrier water;
b. separating the at least one ammonia compound and/or the water of the metabolic water fraction;
c. isolating the separated at least one ammonia compound and/or the water, wherein the water is in the form of pure water.

2. The method according to claim 1, wherein the method includes the step of:
culturing in the bioreactor the methanogenic microorganisms in a suitable liquid culture medium comprising minerals in a continuous process;
redosing at least one fraction of the isolated at least one ammonia compound into the bioreactor and/or recycling the pure water to an electrolyzer.

3. The method of claim 1, wherein the recycling of electrons further comprises:
performing a reductive power regeneration with the isolated pure water by electrolysing the pure water and recycling the regenerated electrons back in the bioreactor, wherein H2 serves as an intermediate electron carrier.

4. The method according to claim 2, wherein the step of culturing the methanogenic microorganisms further comprise:
controlling and regulating the concentration of the at least one ammonia compound in the culture medium to maintain the at least one ammonia compound concentration in the culture medium to be at a given amount of 0.001 to 1.7 M.

5. The method according to claim 2, wherein the step of culturing the methanogenic microorganisms further comprises:
keeping the culture conditions anaerobic or facultatively anaerobic;
optionally stirring the culture; and/or
keeping the temperatures in a range from 5° C. and 95° C.

6. The method according to claim 2, wherein at least one methanogenic microorganism is hydrogenotrophic and is Archaea or archaebacteria comprising Methanobacterium, Methanobrevibacter, Methanothermobacter, Methanococcus, Methanosarcina, Methanopyrus or mixtures thereof.

7. The method according to claim 2, wherein the step of extracting from the culture medium the metabolic water fraction comprises the step of filtrating the at least one ammonia compound and the water from the culture medium and/or comprises the step of evaporating excess water from the culture medium and/or comprises the step of distillation.

8. The method according to claim 7, wherein the step of filtrating the at least one ammonia compound and the water from the culture medium is performed by reverse osmosis using at least one semipermeable membrane for the at least one ammonia compound and the water in contact with the culture medium.

9. The method according to claim 2, wherein the isolated at least one ammonia compound is in the form of NH₃, NH₄OH, (NH₄)HCO₃, (NH₄)₂SO₄ or NH₄Cl or combinations thereof, preferably in the form of NH₄OH.

10. The method according to claim 2, further comprising:
separating at least one entity of the minerals comprised in the metabolic water fraction from the remaining metabolic water components;
optionally storing the separated at least one entity of minerals; and
redosing of the at least one entity of minerals in the bioreactor.

11. The method according to claim 10, wherein the at least one entity of minerals is selected from the group consisting of: iron, nickel, potassium, phosphorus, sodium, chloride, cobalt, selenium, tungsten, magnesium, molybdenum, sulfur, nitrilotriacetate, nitrilotriacetic acid, L-cysteine and resazurin or mixtures thereof.

12. The method according to claim 2, wherein the step of culturing the methanogenic microorganisms further comprise the steps of:
isolating the methanogenic microorganisms comprised in the extracted metabolic water fraction from the remaining metabolic water components;
optionally storing the separated methanogenic microorganisms;
optionally lysing at least fractions of the separated methanogenic microorganisms; and
recycling the separated methanogenic microorganisms and/or optionally lysed fractions thereof back in the culture medium.

13. The method according to claim 2, wherein the step of culturing the methanogenic microorganisms comprises at least one cycle of culturing the methanogenic microorganisms under:
a first phase in a continuous process in a suitable liquid minerals containing culture medium comprising a reduced supply of at least one entity of minerals;
followed by a second phase, characterized by refreshing the culture medium;
optionally followed by a third phase in a continuous process comprising a reduced supply of at least one entity of minerals.

14. The method according to claim 13, wherein the step of culturing the methanogenic microorganisms comprises at least one cycle of culturing the methanogenic microorganisms under:
a fourth phase under cell retention conditions; followed by
a fifth phase, characterized by culturing the cells under no cell retention conditions;
optional followed by a sixth phase under cell retention conditions.

15. The method according to claim 2, wherein the method alternatively comprises:
collecting methane or a methane enriched gas composition and/or at least one other synthesis product from the bioreactor.

16. A biomethanation method in a bioreactor utilizing a culture of methanogenic microorganisms in a culture medium for producing and collecting methane or a methane enriched gas composition comprising the steps of:
i. recycling of at least one ammonia compound and/or;
ii. recycling of electrons;
wherein water (H2O) serves as the carrier for electrons;
including the steps of:
a. culturing in the bioreactor the methanogenic microorganisms in a suitable liquid culture medium comprising minerals in a continuous process;
b. extracting from the culture medium a metabolic water fraction comprising an at least one ammonia compound and the electron carrier water;
c. separating the at least one ammonia compound and/or the water of the metabolic water fraction;
d. isolating the separated at least one ammonia compound and/or the water, wherein the water is in the form of pure water; and
e. redosing at least one fraction of the isolated at least one ammonia compound into the bioreactor and/or recycling the pure water to an electrolyzer,
wherein the recycling of electrons further comprises: performing a reductive power regeneration with the isolated pure water by electrolysing the pure water and recycling the regenerated electrons back in the bioreactor, wherein H2 serves as an intermediate electron carrier.

17. The method according to claim 16, wherein the step of culturing the methanogenic microorganisms further comprise:
controlling and regulating the concentration of the at least one ammonia compound in the culture medium to maintain the at least one ammonia compound concentration in the culture medium to be at a given amount of 0.001 to 1.7 M.

18. The method according to claim 16, wherein at least one methanogenic microorganism is hydrogenotrophic and is Archaea or archaebacteria comprising Methanobacterium, Methanobrevibacter, Methanothermobacter, Methanococcus, Methanosarcina, Methanopyrus or mixtures thereof.

19. The method according to claim 16, wherein (a) the step of extracting from the culture medium the metabolic water fraction comprises the step of filtrating the at least one ammonia compound and the water from the culture medium and/or comprises the step of evaporating excess water from the culture medium and/or comprises the step of distillation; or (b) the step of extracting from the culture medium the metabolic water fraction comprises the step of filtrating the at least one ammonia compound and the water from the culture by reverse osmosis using at least one semipermeable membrane for the at least one ammonia compound and the water in contact with the culture medium.

20. The method according to claim 16, further comprising:
separating at least one entity of the minerals comprised in the metabolic water fraction from the remaining metabolic water components;
optionally storing the separated at least one entity of minerals; and
redosing of the at least one entity of minerals in the bioreactor.
